(19) United States
(12) Patent Application Publication
(10) Pub. No.: US 2018/0030038 A1
(43) Pub. Date: Feb. 1, 2018

(54) Polymorphic Forms of Afatinib Dimaleate

(71) Applicant: Mylan Laboratories Ltd., Panvel (IN)

(72) Inventors: Ramakoteswara Rao Jetti, Panvel (IN); Anjaneyaraju Indukuri, Panvel (IN); Satish Beeravelly, Panvel (IN); Asharani Gorantla, Panvel (IN); B.A. Ramireddy, Panvel (IN); Ruchira M. Sarbajna, Panvel (IN); Sivalakshmidevi Arikatla, Panvel (IN)

(21) Appl. No.: 15/225,015

(22) Filed: Aug. 1, 2016

(51) Int. Cl. C07D 405/12

(52) U.S. Cl. CPC C07D 405/12; C07B 2200/13

(57) ABSTRACT

The present disclosure relates to amorphous and crystalline forms of 2-butenamide, N-[4-[(3-chloro-4-fluorophenyl)amino]7-[[(3S)-tetrahydro-3-furanyl]oxy]-6-quinazolinyl]-4-(dimethyl amino)-,(2E)-, (2Z)-2-butenedioate (1:2) (afatinib dimaleate). The present disclosure also relates to process for the preparation of amorphous and crystalline forms of afatinib dimaleate. Further, the present disclosure relates to solid dispersion of amorphous afatinib dimaleate and the crystalline form of afatinib.

The specification below is a coagnate application of Indian Patent Application no. 1846/MUM/2014 dated Jun. 4, 2014, Indian Patent Application no. 2275/MUM/2014 dated Jul. 11, 2014, Indian Patent Application no. 2561/MUM/2014 dated Aug. 8, 2014 and Indian Patent Application no. 3140/MUM/2014 dated Oct. 1, 2014.

### CROSS-REFERENCE TO RELATED APPLICATIONS

This application claims the benefit of the earlier filing date of Indian Provisional Patent Application No. 1846/MUM/2014 filed on Jun. 4, 2014, Indian Provisional Patent Application No. 2275/MUM/2014 filed on Jul. 11, 2014, Indian Provisional Patent Application No. 2561/MUM/2014 filed on Aug. 8, 2014, and Indian Provisional Patent Application No. 3140/MUM/2014 filed on Oct. 1, 2014.

### BACKGROUND OF THE INVENTION

### Field of the Invention

The present invention relates generally to amorphous and crystalline forms of afatinib dimaleate and afatinib free base and processes for the preparation thereof.

### Background of the Invention

Afatinib dimaleate, chemically known as 2-butenamide, N-[4-[(3-chloro-4-fluoropheny)amino]7-[[(3S)-tetrahydro-3-furanyl]oxy]-6-quinazolinyl]-4-(dimethyl amino)-,(2E)-, (2Z)-2-butenedioate (1:2), is an orally administered irreversible inhibitor of the epidermal growth factor receptor (EGFR) and human epidermal receptor 2 (HER2) tyrosine kinases. Afatinib dimaleate is marketed under the brand name GILOTRIF®, which is indicated for treatment of patients with several solid tumors including non-small cell lung cancer (NSCLC), breast, head and neck cancer, and a variety of other cancers.

The structural formula of afatinib dimaleate is:

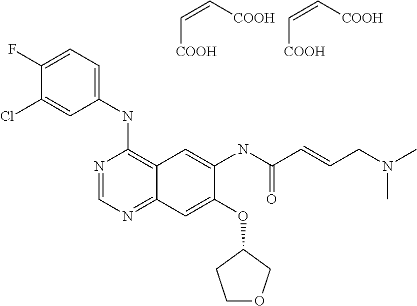 [SMILES: CN(C)C/C=C/C(=O)Nc1cc2c(Nc3ccc(F)c(Cl)c3)ncnc2cc1O[C@H]1CCOC1 ; O=C(O)/C=C\C(=O)O ; O=C(O)/C=C\C(=O)O]

The present invention provides novel crystalline and amorphous forms and process for the preparation of afatinib dimaleate. The present invention also provides a crystalline form of afatinib. The present invention further provides a solid dispersion preparation of afatinib dimaleate.

### SUMMARY OF THE INVENTION

A first aspect of the present disclosure is to provide a novel crystalline afatinib dimaleate form-M1, which is characterized by powdered X-ray diffraction pattern as shown in FIG. 1.

Another aspect of the present disclosure is to provide a process for the preparation of crystalline afatinib dimaleate form-M1 comprising the steps of:
a) dissolving afatinib and maleic acid in an organic solvent;
b) adding an anti-solvent; and
c) isolating crystalline afatinib dimaleate form-M1.

Yet another aspect of the present disclosure is to provide a process for the preparation of crystalline afatinib dimaleate form-M1 comprising the steps of:
a) dissolving afatinib dimaleate in an organic solvent;
b) adding an anti-solvent; and
c) isolating crystalline afatinib dimaleate form-M1.

A further aspect of the present disclosure is to provide a novel crystalline afatinib dimaleate form-M2.

One aspect of the present disclosure is to provide a novel crystalline afatinib dimaleate form-M2, which is characterized by powdered X-ray diffraction pattern as shown in FIG. 2.

Another aspect of the present disclosure is to provide a process for the preparation of crystalline afatinib dimaleate form-M2 comprising drying the crystalline afatinib dimaleate form-M1 at 35-55° C. for 15-24 hours.

A further aspect of the present disclosure is to provide a novel crystalline afatinib dimaleate form-M3, which is characterized by powdered X-ray diffraction pattern as shown in FIG. 3.

Another aspect of the present disclosure is to provide a process for the preparation of crystalline afatinib dimaleate form-M3 comprising the steps of:
a) dissolving afatinib and maleic acid in an organic solvent;
b) adding an anti-solvent; and
c) isolating crystalline afatinib dimaleate form-M3.

An additional aspect of the present disclosure is to provide a novel crystalline afatinib dimaleate form-M4, which is characterized by the powdered X-ray diffraction pattern as shown in FIG. 4.

Another aspect of the present disclosure is to provide a process for the preparation of crystalline afatinib dimaleate form-M4 comprising the steps of:
a) dissolving afatinib in an organic solvent;
b) adding maleic acid solution to the solution obtained in step (a); and
c) isolating crystalline afatinib dimaleate form-M4.

A further aspect of the present disclosure is to provide a novel crystalline afatinib dimaleate farm-M5, which is characterized by the powdered X-ray diffraction pattern as shown in FIG. 5.

Another aspect of the present disclosure is to provide a process for the preparation of crystalline afatinib dimaleate form-M5 comprising the steps of:
a) dissolving afatinib in an organic solvent;
b) adding maleic acid solution to the solution obtained in step (a); and
c) isolating crystalline afatinib dimaleate form-M5.

An additional aspect of the present disclosure is to provide a novel crystalline afatinib dimaleate form-M6, which is characterized by powdered X-ray diffraction pattern as shown in FIG. 6.

Another aspect of the present disclosure is to provide a process for the preparation of crystalline afatinib dimaleate form-M6 comprising the steps of:
a) dissolving afatinib in an organic solvent;
b) optionally seeding with afatinib dimaleate form-M6;
c) adding maleic acid solution; and
d) isolating crystalline afatinib dimaleate form-M6 by filtration, washing and drying.

Another aspect of the present disclosure is to provide a novel crystalline afatinib dimaleate form-M7.

One aspect of the present disclosure is to provide a novel crystalline afatinib dimaleate form-M7, which is characterized by powdered X-ray diffraction pattern as shown in FIG. 7.

Another aspect of the present disclosure is to provide a process for the preparation of crystalline afatinib dimaleate form-M7 comprises heating the crystalline afatinib dimaleate form-M6 at 80-90° C. for 1-2 hours.

In another aspect, the present disclosure provides a process for the preparation of crystalline afatinib dimaleate farm-M7 comprises grinding the crystalline afatinib dimaleate form-M6.

An additional aspect of the present disclosure is to provide a novel crystalline afatinib dimaleate form-M8, which is characterized by powdered X-ray diffraction pattern as shown in FIG. 8.

Another aspect of the present disclosure is to provide a process for the preparation of crystalline afatinib dimaleate form-M8 comprising the steps of:
a) dissolving afatinib in an organic solvent;
b) optionally seeding with afatinib dimaleate form-M8;
c) adding maleic acid solution; and
d) isolating crystalline afatinib dimaleate form-M8 by filtration, washing and drying.

In another aspect, the present disclosure provides a process for the preparation of crystalline afatinib dimaleate form-M8 comprises heating the crystalline afatinib dimaleate form-M6 at approximately 40-50′C for about 30-60 minutes.

A further aspect of the present disclosure is to provide a novel crystalline afatinib dimaleate form-M9, which is characterized by powdered X-ray diffraction pattern as shown in FIG. 9.

Another aspect of the present disclosure is to provide a process for the preparation of crystalline afatinib dimaleate form-M9 comprising the steps of:
a) dissolving afatinib in an organic solvent;
b) optionally seeding with afatinib dimaleate form-M9;
c) adding maleic acid solution; and
d) isolating crystalline afatinib dimaleate form-M9 by filtration, washing, and drying.

A further aspect of the present disclosure is to provide an amorphous afatinib dimaleate, which is characterized by powdered X-ray diffraction pattern as shown in FIG. 10.

Another aspect of the present disclosure is to provide a process for the preparation of amorphous afatinib dimaleate comprising the steps of:
a) dissolving afatinib dimaleate in an organic solvent;
b) removing the solvent from step (a); and
c) isolating amorphous afatinib dimaleate.

Yet another aspect of the present disclosure is to provide a process for the preparation of amorphous afatinib dimaleate comprising the steps of:
a) dissolving afatinib and maleic acid in an organic solvent;
b) removing the solvent from step (a); and
c) isolating amorphous afatinib dimaleate.

An additional aspect of the present disclosure is to provide a solid dispersion of amorphous afatinib dimaleate together with one or more pharmaceutically acceptable carriers.

One aspect of the present disclosure is to provide a process for the preparation of solid dispersion of amorphous afatinib dimaleate comprising the steps of:
a) dissolving afatinib, maleic acid and one or more pharmaceutically acceptable carriers in an organic solvent;
b) adding an anti-solvent; and
c) isolating solid dispersion of amorphous afatinib dimaleate.

A further aspect of the present disclosure is to provide a novel crystalline afatinib form-M, which is characterized by powdered X-ray diffraction pattern as shown in FIG. 11.

Another aspect of the present disclosure is to provide a process for the preparation of crystalline afatinib form-M comprising the steps of:
a) dissolving afatinib in an organic solvent;
b) adding an anti-solvent; and
c) isolating crystalline afatinib form-M.

### BRIEF DESCRIPTION OF THE FIGURES

Further aspects of the present disclosure together with additional features contributing thereto and advantages accruing there from will be apparent from the following description of embodiments of the disclosure which are shown in the accompanying drawing figures wherein:

FIG. 1 is an X-ray powder diffractogram of crystalline form-M1 of afatinib dimaleate;

FIG. 2 is an X-ray powder diffractogram of crystalline form-M2 of afatinib dimaleate;

FIG. 3 is an X-ray powder diffractogram of crystalline form-M3 of afatinib dimaleate;

FIG. 4 is an X-ray powder diffractogram of crystalline form-M4 of afatinib dimaleate;

FIG. 5 is an X-ray powder diffractogram of crystalline form-M5 of afatinib dimaleate;

FIG. 6 is an X-ray powder diffractogram of crystalline form-M6 of afatinib dimaleate; FIG. 7 is an X-ray powder diffractogram of crystalline form-M7 of afatinib dimaleate;

FIG. 8 is an X-ray powder diffractogram of crystalline form-M8 of afatinib dimaleate;

FIG. 9 is an X-ray powder diffractogram of crystalline form-M9 of afatinib dimaleate;

FIG. 10 is an X-ray powder diffractogram of amorphous afatinib dimaleate; and

FIG. 11 is an X-ray powder diffractogram of crystalline form-M of afatinib.

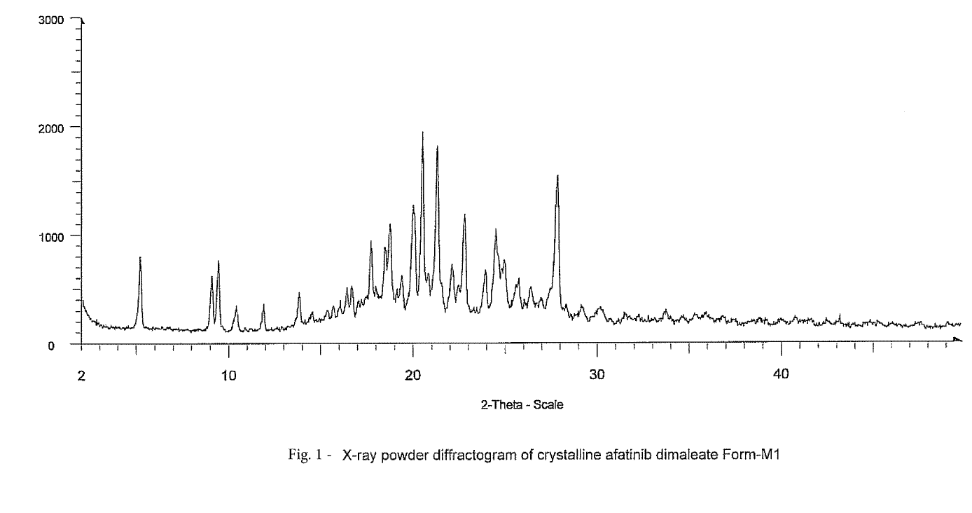

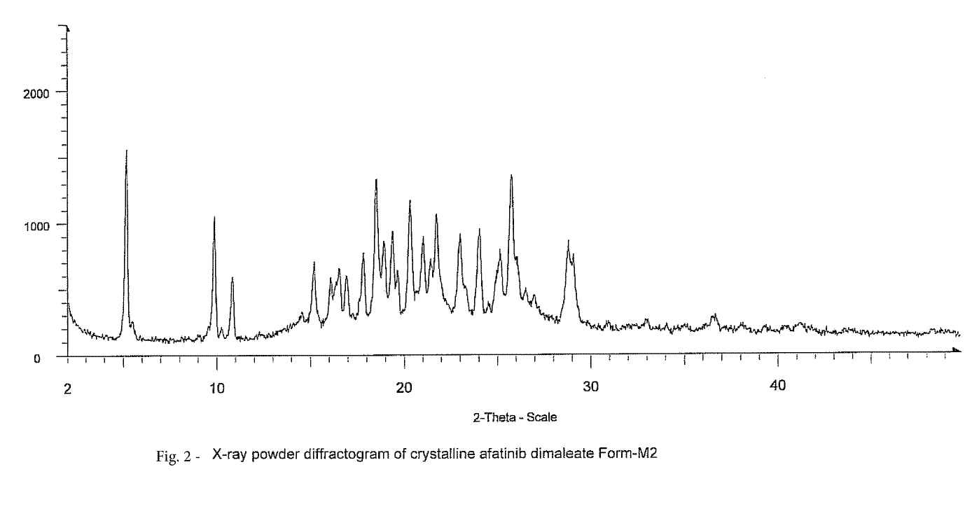

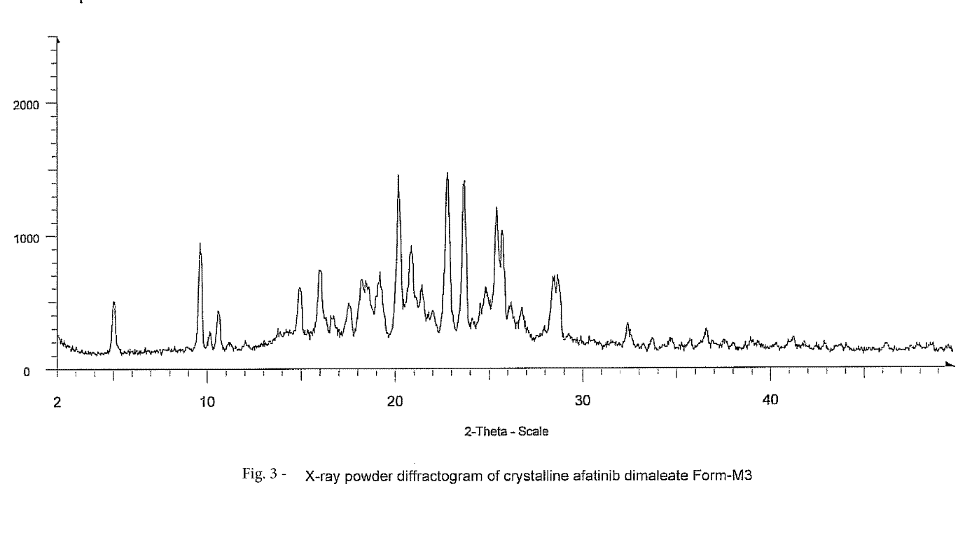

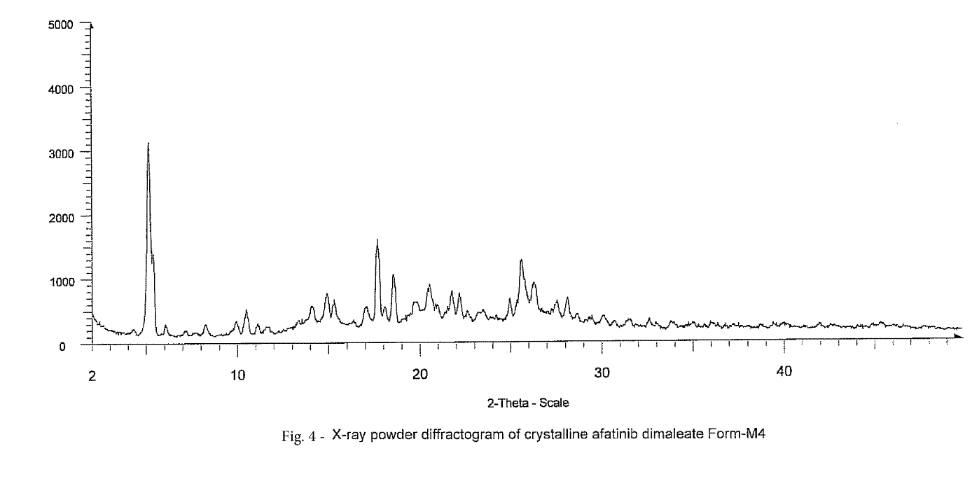

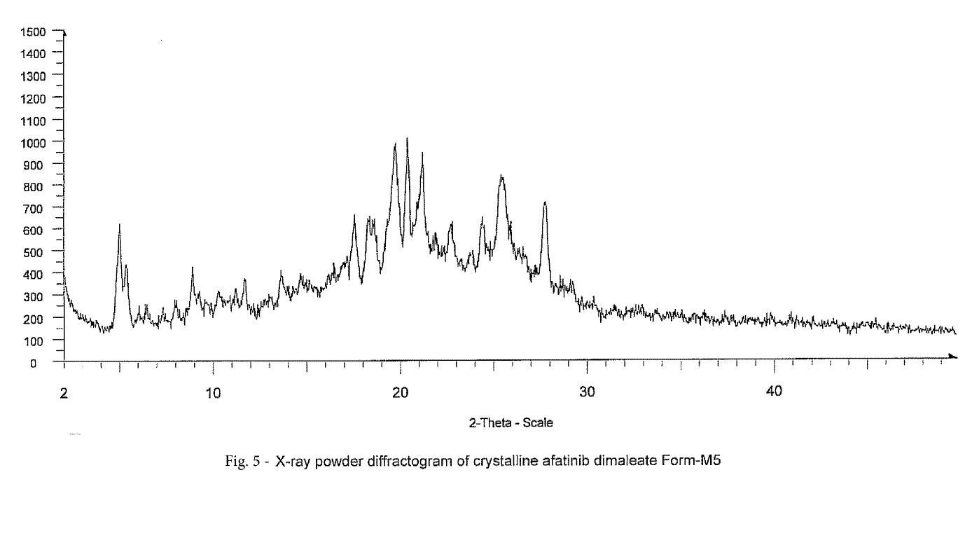

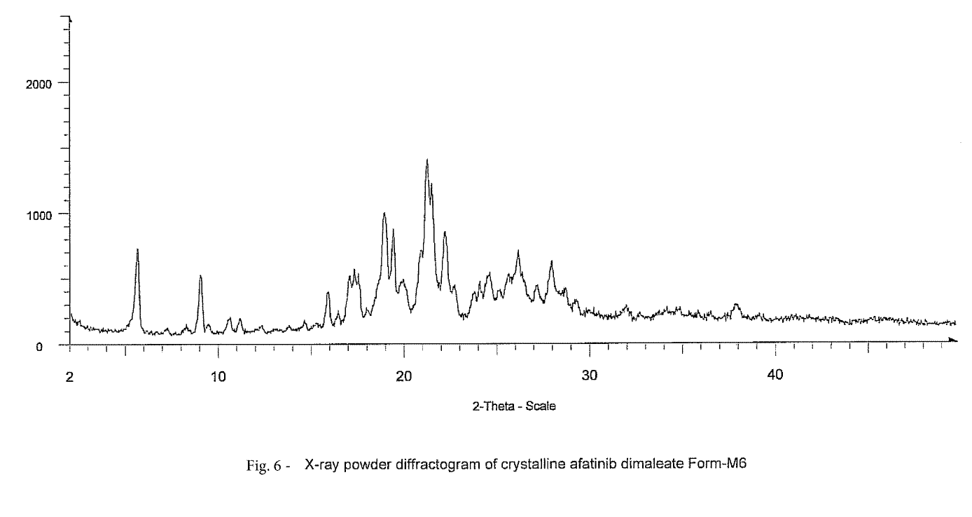

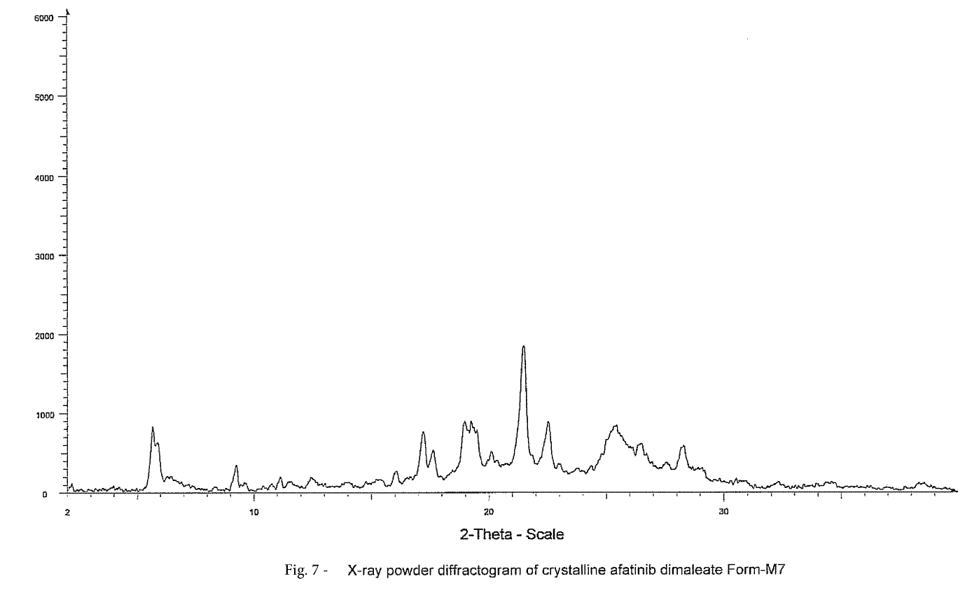

### DETAILED DESCRIPTION OF THE DISCLOSURE

It is to be understood that the description of the present invention has been simplified to illustrate elements that are relevant for a clear understanding of the invention, while eliminating, for purposes of clarity, other elements that may be well known.

The present disclosure provides amorphous and crystalline forms, and solid dispersion, of afatinib dimaleate. The present disclosure also relates to crystalline form of afatinib and a process for the preparation of amorphous and crystalline forms of afatinib dimaleate.

The polymorph of the present disclosure may be characterized by their X-ray powder diffraction (“PXRD”) patterns. Thus, the PXRD patterns of the polymorphs of the disclosure were measured on BRUKER D-8 DISCOVER powder dlffractometer equipped with goniometer of θ/2θ configuration and LYNX EYE detector. The Cu-anode X-ray tube was operated at 40 kV and 30 mA. The experiments were conducted over the 2θ range of 2.0°-50.0°, 0.030° step size and 0.4 seconds step time.

One aspect of the present invention provides crystalline afatinib dimaleate form-M1. According to the present invention, crystalline afatinib dimaleate form-M1 may be characterized by a PXRD pattern having significant peaks at 2θ angle positions of about 5.07, 20.43, 21.22, 22.73, and 27.78±0.2°.

Within the context of the present invention, crystalline afatinib dimaleate form-M1 may be further characterized by a PXRD pattern having significant peaks at 2θ angle positions of about 5.07, 8.98, 9.34, 10.30, 11.79, 13.73, 14.43, 15.26, 16.30, 16.60, 17.05, 17.62, 18.40, 18.64, 19.40, 19.95, 20.43, 20.74, 21.22, 22.02, 22.73, 23.85, 24.41, 24.90, 25.64, 26.32, 26.86, 27.33, and 27.77±0.2°.

One embodiment of the present invention provides a process for the preparation of crystalline afatinib dimaleate form-M1 which includes the following steps:
a) dissolving afatinib and maleic acid in an organic solvent;
b) adding an anti-solvent; and
c) isolating crystalline afatinib dimaleate form-M1.

According to the present disclosure, afatinib and maleic acid are dissolved in an organic solvent. Within the context of the present invention, this step may be carried out at a temperature of about 60° C. to about 75° C. Examples of suitable organic solvents include dimethyl formamide. In some embodiments of the present invention, dimethyl formamide was found to be a particularly useful organic solvent.

The solution may then be cooled to a temperature of about 20° C. to about 35° C. Next, an anti-solvent may be added and the solution may be stirred, forming a solid. Within the context of the present invention, the anti-solvent employed may be, for example, an ether solvent, a hydrocarbon solvent, or mixtures thereof. Examples of suitable ether solvents include diethyl ether, di-isopropyl ether, and methyl tent-butyl ether. Examples of suitable hydrocarbon solvents include n-hexane and n-heptane. One of skill in the art will recognize other ether solvents and hydrocarbon solvents that may be employed. In some embodiments, methyl tert-butyl ether has been found to be a particularly useful anti-solvent.

Next, the crystalline afatinib dimaleate form-M1 may be isolated using methods well known in the art, including, for example, by filtering the solution and drying the solid at a temperature of about 35° C. to about 45° C. under vacuum for 5-6 hours.

Yet another embodiment of the present disclosure is a process for the preparation of crystalline afatinib dimaleate Form-M1 comprising the steps of:
a) dissolving afatinib dimaleate in an organic solvent,
b) adding anti-solvent and
c) isolating crystalline afatinib dimaleate Form-M1.

According to the present disclosure, afatinib dimaleate is dissolved in an organic solvent at a temperature of about 60° C. to about 75° C. and then cooled the solution to a temperature of about 20° C. to about 35° C. Examples of suitable organic solvents include dimethyl formamide. In some embodiments of the present invention, dimethyl formamide was found to be a particularly useful organic solvent. To the clear solution anti-solvent is added and then stirred. Within the context of the present invention, the anti-solvent employed may be, for example, an ether solvent, a hydrocarbon solvent, or mixtures thereof. Examples of suitable ether solvents include diethyl ether, di-isopropyl ether, and methyl tert-butyl ether. Examples of suitable hydrocarbon solvents include n-hexane and n-heptane. One of skill in the art will recognize other ether solvents and hydrocarbon solvents that may be employed. In some embodiments, methyl tert-butyl ether has been found to be a particularly useful anti-solvent.

Next, the crystalline afatinib dimaleate form-M1 may be isolated using methods well known in the art, including, for example, by filtering the solution and drying the solid at a temperature of about 35° C. to about 45° C. under vacuum for 1-2 hours.

Another aspect of the present invention provides crystalline afatinib dimaleate form-M2. According to the present invention, crystalline afatinib dimaleate form-M2 may be characterized by a PXRD pattern having significant peaks at 2θ angle positions of about 5.12, 9.83, 18.51, 19.38, 20.32, 21.74, and 25,76±0.2°.

Within the context of the present invention, crystalline afatinib dimaleate form-M2 may be further characterized by a PXRD pattern having significant peaks at 2θ angle positions of about 5.12, 5.44, 9.83, 10.23, 10.79, 14.54, 15.18, 16.08, 16.51, 16.91, 17.81, 18.51, 18.91, 19.38, 19,66, 20.32, 21.02, 21.43, 21.74, 23.0, 23.31, 24.04, 24.54, 24.95, 25.16, 25.76, 26.07, 26.52, 26.98, 28.81, 29.06, 30.95, 33.02, 34.11, and 36.69±0.2°.

Another embodiment of the present invention provides a process for the preparation of crystalline afatinib dimaleate form-M2 which may be achieved by drying the crystalline afatinib dimaleate form-M1 at about 35° C. to about 55° C. for about 15 to about 24 hours.

In some embodiments of the present invention, drying crystalline afatinib dimaleate form-M1 under vacuum at about 40° C. for about 15 to about 24 hours was particularly useful in obtaining crystalline afatinib dimaleate form-M2.

Another aspect of the present invention provides crystalline afatinib dimaleate form-M3. According to the present invention, crystalline afatinib dimaleate form-M3 may be characterized by a PXRD pattern having significant peaks at 2θ angle positions of about 9.61, 15.98, 18.20, 20.19, 20.85, 22.79, and 23.68±0.2°.

Within the context of the present invention, crystalline afatinib dimaleate form-M2 may be further characterized by a PXRD pattern having significant peaks at 2θ angle positions of about 4.98, 8.91, 9.61, 10.11, 10.59, 11.15, 12.02, 14.91, 15.98, 16.64, 17.53, 18.20, 18.43, 19.17, 20.19, 21.41, 22.0, 22.79, 23.68, 24.56, 24.85, 25.40, 25.72, 26.15, 26.75, 28.46, 28.69, 32.43, 33.27, 33.73, 34.73, 35.76, 36.60, 37.55, 38.04, 38.99, 40.31, 41.25, 42.48, 42.92, 46.24, and 46.62±0.2°.

Another embodiment of the present invention provides a process for the preparation of crystalline afatinib dimaleate form-M3, which includes the following steps:
a) dissolving afatinib and maleic acid in an organic solvent;
b) adding an anti-solvent; and
c) isolating crystalline afatinib dimaleate form-M3.

According to the present disclosure, afatinib and maleic acid are dissolved in an organic solvent. Within the context of the present invention, this may be carried out at a temperature of about 25° C. to about 30° C. Within the context of the present invention, suitable organic solvents include, for example, 1-methyl 2-pyrrolidinone. In some embodiments of the present invention, 1-methyl 2-pyrrolidinone was found to be a particularly useful organic solvent.

Next, an anti-solvent is added and the solution is stirred. Within the context of the present invention, the anti-solvent may be a ketone, for example, acetone, methyl ethyl ketone, methyl isobutyl ketone, ethyl isopropyl ketone, or mixtures thereof. In some embodiments, acetone was found to be a particularly useful anti-solvent. One of skill in the art will recognize other ketone solvents that may be employed.

Crystalline afatinib dimaleate form-M3 may then be isolated using methods well known in the art, including, for example, by filtering the solution and drying the obtained solid at a temperature of about 40° C. to about 50° C. under vacuum for about 2 to about 3 hours.

Another aspect of the present invention provides crystalline afatinib dimaleate form-M4. According to the present invention, crystalline afatinib dimaleate form-M4 may be characterized by a PXRD pattern having significant peaks at 2θ angle positions of about 5.01, 17.66, 18.53, 20.49 and 25.58±0.2°.

Within the context of the present invention, crystalline afatinib dimaleate form-M4 may be further characterized by a PXRD pattern having significant peaks at 2θ angle positions of about 4.24, 5.01, 5.35, 6.01, 7.12, 8.22, 9.90, 10.44, 11.08, 11.62, 14.06, 14.88, 15.26, 17.01, 17.66, 18.05, 18.53, 19.71, 20.49, 21.73, 22.17, 22,60, 24.95, 25.58, 26.28, 27.53, 28.1, 28.63, 29.37, 30.09, 30.69, 31.52, and 32.59±0.2°.

Another embodiment of the present invention provides a process for the preparation of crystalline afatinib dimaleate form-M4 which includes the following steps:
a) dissolving afatinib in an organic solvent to create a solution;
b) adding maleic acid solution to the solution above; and
c) isolating crystalline afatinib dimaleate form-M4.

According to this embodiment of the present disclosure, afatinib is dissolved in an organic solvent. Next, a solution of maleic acid is added. The solution may be then stirred at a temperature of about 25° C. to about 30° C. for about 17 to about 18 hours. Crystalline afatinib dimaleate form-M4 may then be isolated using methods well known in the art, including, for example, by filtering the solution and drying the solid at a temperature of about 40° C. to about 50° C. under vacuum for about 3 to about 4 hours.

Within the context of the present invention, the organic solvent used to dissolve afatinib may be a ketone, for example, acetone, methyl ethyl ketone, methyl isobutyl ketone, ethyl isopropyl ketone, or mixtures thereof. In some embodiments of the invention methyl ethyl ketone was found to be a particularly useful organic solvent. One of skill in the art will recognize other ketone solvents that may be employed.

Within the context of the present invention, the maleic acid solution may be prepared by dissolving maleic acid in the same organic solvent as used to dissolve afatinib in the first step for the process for the preparation of afatinib dimaleate form-M4.

Another aspect of the present invention provides crystalline afatinib dimaleate form-M5. According to the present invention, crystalline afatinib dimaleate form-M5 may be characterized by a PXRD pattern having significant peaks at 2θ angle positions of about 4.95, 17.53, 18.29, 18.55, 19.69, 20.37, 21.15, and 27.74±0.2°.

Within the context of the present invention, crystalline afatinib dimaleate form-M5 may be further characterized by a PXRD pattern having significant peaks at 2θ angle positions of about 4.95, 5.32, 5.98, 6.38, 7.25, 7.95, 8.87, 10.25, 11.17, 11.65, 12.97, 13.61, 14.57, 16.40, 17.53, 18.29, 18.55, 19.69, 20.37, 21.15, 21.83, 22.68, 23.81, 24.39, 25.42, 27.74, and 29.19±0.2°.

One embodiment of the present invention provides a process for the preparation of crystalline afatinib dimaleate form-M5, which includes the following steps:
a) dissolving afatinib in an organic solvent to create a solution;
b) adding a maleic acid solution to the solution above; and
c) isolating crystalline afatinib dimaleate form-M5.

According to this embodiment of the present disclosure, afatinib is dissolved in an organic solvent. Within the context of the present invention, the organic solvent may be a ketone. Examples of suitable ketones include acetone, methyl ethyl ketone, methyl isobutyl ketone, ethyl isopropyl ketone, and mixtures thereof. In some embodiments, acetone was found to be a particularly useful organic solvent. One of skill in the art will recognize other ketone solvents that may be employed.

Next, a solution of maleic acid is added. The reaction may then be stirred for about 17 to about 18 hours at 25-35° C. Crystalline afatinib dimaleate form-M5 may then be isolated using methods well known in the art, including, for example, by filtering the solution and drying the obtained solid at a temperature of about 40° C. to about 50° C. under vacuum for about 3 to about 4 hours.

Another aspect of the present invention provides crystalline afatinib dimaleate form-M5. According to the present invention, crystalline afatinib dimaleate form-M6 may be characterized by a PXRD pattern having significant peaks at 2θ angle positions of about 5.59, 18.94, 19.41, 21.23, 21.48, 22.19, 26.13, 27.95±0.2°.

Within the context of the present invention, crystalline afatinib dimaleate form-M6 may be further characterized by a PXRD pattern having significant peaks at 2θ angle positions of about 5.59, 7.21, 9.02, 9.43, 10.57, 11.14, 12.27, 13.78, 14.63, 15.87, 16.44, 17.04, 17.51, 18.00, 18.94, 19.41, 19.90, 20.89, 21.23, 21.48, 22.19, 22.69, 23.78, 24.06, 24.55, 25.10, 25.63, 26.13, 27.15, 27.95, 28.68, 29.28, 31.96, 34.84, and 37.93±0.2°.

One embodiment of the present invention provides a process for the preparation of crystalline afatinib dimaleate form-M6 which includes the following steps:
a) dissolving afatinib in an organic solvent;
b) optionally seeding with afatinib dimaleate form-M6;
c) adding maleic acid solution; and
d) isolating crystalline afatinib dimaleate form-M6.

According to this embodiment of the present disclosure, afatinib may be dissolved in an organic solvent at a temperature of about 25° C. to about 30° C., and then cooled to about 0° C. to about 5° C. Maleic acid (which may be in the form, for example, of maleic acid dissolved in the same organic solvent) may then be added at the same temperature, and the solution may then be stirred. Next, the solution may optionally be seeded with afatinib dimaleate form-M6.

The organic solvent used to dissolve afatinib (and for making the maleic acid solution) may be, for example, acetone.

Crystalline afatinib dimaleate form-M6 may then be isolated using methods well known in the art, including, for example, by filtering the solution and drying the obtained solid at about 35° C. to about 45° C. under vacuum for about 3 to about 4 hours.

Another aspect of the present invention provides crystalline afatinib dimaleate form-M7. According to the present invention, crystalline afatinib dimaleate form-M7 may be characterized by a PXRD pattern having significant peaks at 2θ angle positions of about 5.63, 17.19, 18.96, 21.47, 22.53, and 25.39±0.2°.

Within the context of the present invention, crystalline afatinib dimaleate form-M7 may be further characterized by a PXRD pattern having significant peaks at 2θ angle positions of about 5.63, 5.86, 6.25, 8.29, 9.19, 9.60, 10.38, 10.72, 11.09, 11.50, 12.43, 13.94, 15.36, 16.03, 17.19, 17.61, 18.96, 19.25, 19.42, 20.09, 21.47, 22.53, 23.01, 25.39, 26.48, 27.59, 28.27, 29.08, 32.34, 34.66, 36.21, 37.78, and 38.35±0.2°.

One embodiment of the present invention provides a process for the preparation of crystalline afatinib dimaleate form-M7 which may be achieved by heating the crystalline afatinib dimaleate form-M6 at about 80° C. to about 90° C. for about 1 to about 2 hours.

Another embodiment of the present disclosure provides a process for the preparation of crystalline afatinib dimaleate form-M7 which can be achieved by grinding the crystalline afatinib dimaleate form-M6.

Another aspect of the present invention provides crystalline afatinib dimaleate form-M5. According to the present invention, crystalline afatinib dimaleate form-M8 may be characterized by a PXRD pattern having significant peaks at 2θ angle positions of about 5.54, 17.17, 17.50, 18.91, 19.34, 21.43, and 22.60±0.2°.

Within the context of the present invention, crystalline afatinib dimaleate form-M8 may be further characterized by a PXRD pattern having significant peaks at 2θ angle positions of about 5.54, 8.28, 9.04, 9.42, 10.57, 11.02, 12.25, 14.64, 15.86, 17.17, 17.50, 18.91, 19.34, 20,04, 21.43, 22.60, 24.73, 26.48, 28.17, and 28.97±0.2°.

One embodiment of the present invention provides a process for the preparation of crystalline afatinib dimaleate form-M8 which includes the following steps:
a) dissolving afatinib in an organic solvent;
b) optionally seeding with afatinib dimaleate form-M8;
c) adding maleic acid solution; and
d) isolating crystalline afatinib dimaleate form-M8.

According to this embodiment of the present disclosure, afatinib is dissolved in an organic solvent a temperature of about 25° C. to about 30° C. Next, maleic acid solution may be added, which may be prepared, for example, by dissolving maleic acid in the same organic solvent.

After half of the maleic acid solution is added, seeds of afatinib dimaleate form-M8 may be optionally added to the solution before adding the remaining maleic acid solution. Crystalline afatinib dimaleate form-M8 may then be isolated using methods well known in the art, including, for example, by filtering the solution and drying the solid at about 50° C. to about 60° C. under vacuum for about 17 to about 18 hours.

Within the context of the present invention, the organic solvent used to dissolve afatinib may be, for example, acetone.

Alternatively, the process for the preparation of crystalline afatinib dimaleate form-M8 may be carried out by dissolving afatinib in an organic solvent at about 25° C. to about 30° C., cooling the solution to a temperature of about 0° C. to about 5° C., and then seeding the solution with afatinib dimaleate form-M8. A maleic acid solution (which may be prepared, for example, by dissolving maleic acid in the same organic solvent) is then added maintaining the temperature of about 0° C. to about 5° C. and the reaction may then be stirred for 5-6 hours.

Within the context of the present invention, the organic solvent used to dissolve afatinib (and preparing the maleic acid solution) may be acetone.

Crystalline afatinib dimaleate form-M8 may then be isolated using methods well known in the art, including, for example, by filtering the solution and drying the resulting solid at about 35° C. to about 45° C. under vacuum for about 17 to about 18 hours.

Another embodiment of the present invention provides a process for the preparation of crystalline afatinib dimaleate form-M8 which may be achieved by heating the crystalline afatinib dimaleate form-M6 at about 40° C. to about 50° C. for about 30 to about 60 minutes.

Another aspect of the present invention provides crystalline afatinib dimaleate form-M9. According to the present invention, crystalline afatinib dimaleate form-M9 may be characterized by a PXRD pattern having significant peaks at 2θ angle positions of about 15.87, 17.41, 19.26, 21.58, 22.57, 24.34, 25.56, and 28.06±0.2°.

Within the context of the present invention, crystalline afatinib dimaleate form-M9 may be further characterized by a PXRD pattern having significant peaks at 2θ angle positions of about 5.38. 6.12, 8.98, 9.92, 10.31, 10.56, 12.07, 13.55, 15.50, 15.87, 17.41, 18.03, 18.54, 18.79, 19.26, 19.68, 20.44, 21.01, 21.58, 22,11, 22.57, 22.88, 24.06, 24.34, 24.87, 25.26, 25.73, 26.37, 26.74, 27.52, 28.06, 28.94, 29.29, 29.53, 31.23, 31.53, 32.30, 33.73, 34.49, 35.34, 35.77, 36.99, 38.27, 39.41, 39.76, 41.46, 43.00, 44.81, 45.92, 47.24, and 46.42±0.2°.

Another embodiment of the present invention provides a process for the preparation of crystalline afatinib dimaleate form-M9 which includes the following steps:
a) dissolving afatinib in an organic solvent;
b) optionally seeding with afatinib dimaleate form-M9;
c) adding maleic acid solution; and
d) isolating crystalline afatinib dimaleate form-M9.

According to this embodiment of the present disclosure, afatinib is dissolved in an organic solvent at a temperature of about 25° C. to about 30° C. Next, maleic acid solution, which may be prepared by dissolving maleic acid in the same organic solvent, is added to the solution and the solution may be stirred.

Within the context of the present invention, this step may be carried out and the organic solvent may be, for example, tetrahydrofuran.

Crystalline afatinib dimaleate form-M9 may then be isolated using methods well known in the art, including, for example, by filtering the solution and drying the solid at about 35° C. to about 45° C. under vacuum for about 5 hours.

Alternatively, the process for the preparation of crystalline afatinib dimaleate form-M9 may be carried out by dissolving afatinib in an organic solvent at about 25° C. to about 30° C. Next, a maleic acid solution, which may be prepared by dissolving maleic acid in the same organic solvent, may be added at the same temperature of about 25° C. to about 30° C. The solution may then be seeded with afatinib dimaleate form-M9.

Within the context of the present invention, the organic solvent used to dissolve afatinib (and preparing the maleic acid solution) may be, for example, tetrahydrofuran.

Crystalline afatinib dimaleate form-M9 may then be isolated using methods well known in the art, including, for example, by filtering the solution and drying the solid at about 45° C. to about 55° C. under vacuum for about 17 to about 18 hours to yield crystalline afatinib dimaleate form-M9.

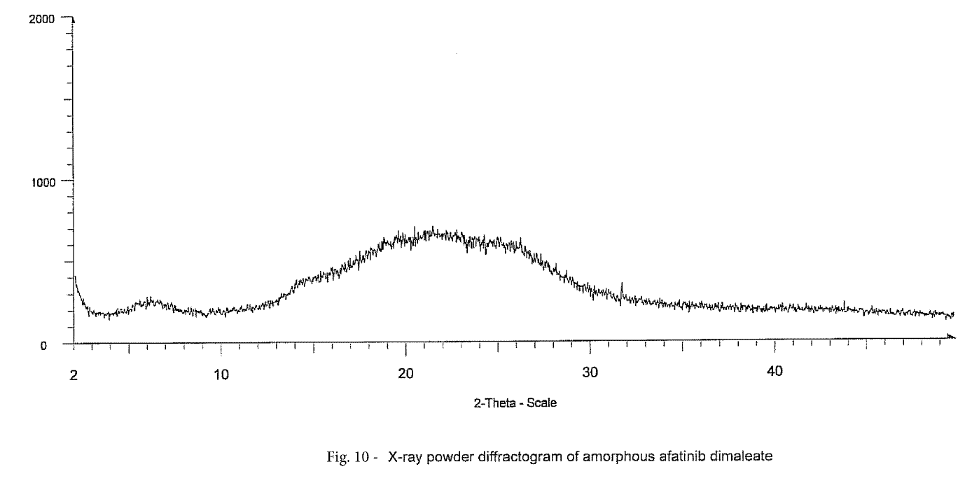

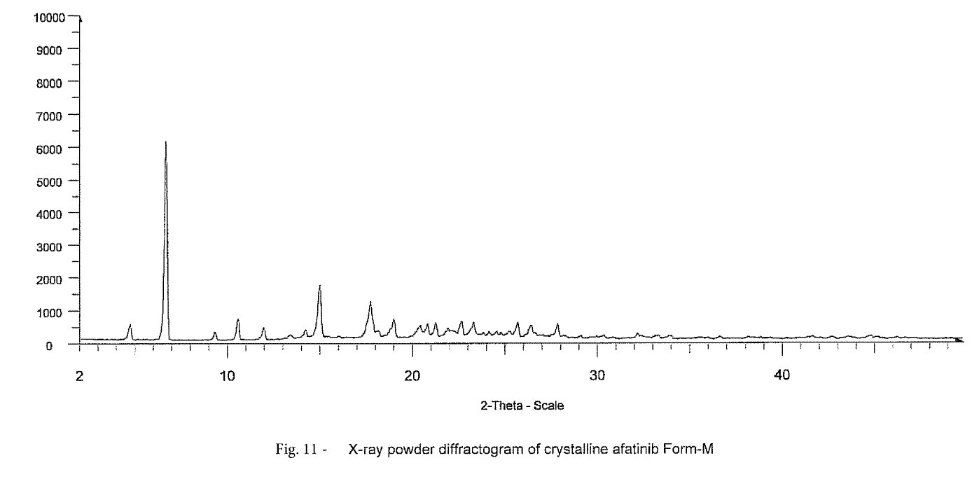

Another aspect of the present invention provides amorphous afatinib dimaleate. Within the context of the present invention, amorphous afatinib dimaleate may characterized by PXRD pattern as shown in FIG. 10.

One embodiment of the present invention provides a process for the preparation of amorphous afatinib dimaleate, which includes the following steps:
a) dissolving afatinib dimaleate in an organic solvent;
b) removing the organic solvent; and
c) isolating amorphous afatinib dimaleate.

According to this embodiment of the present disclosure, afatinib dimaleate is dissolved in an organic solvent. Within the context of the present invention, this step may occur at a temperature of about 50° C. to about 70° C. The organic solvent may be an alcohol, for example, methanol, ethanol, isopropanol, 1-butanol, 2-butanol, isoamyl alcohol, isobutyl alcohol, 1-pentanol, 1-propanol, 2-propanol, or mixtures thereof. One of skill in the art will recognize other alcoholic solvents that may be employed.

The solution may then be filtered, for example, through a Hyflo bed. The solution may then be cooled to a temperature of about 20° C. to about 35° C.

Next, the organic solvent may be removed, for example, by distillation, spray drying, or agitated thin film drying (ATFD) to yield the amorphous form of afatinib dimaleate.

Another embodiment of the present invention provides a process for the preparation of amorphous afatinib dimaleate which includes the following steps:
a) dissolving afatinib and maleic acid in an organic solvent;
b) removing the organic solvent; and
c) isolating amorphous afatinib dimaleate.

According to this embodiment of the present disclosure, afatinib and maleic acid are dissolved in an organic solvent. Within the context of the present invention, this step may be carried out at a temperature of about 50° C. to about 70° C. Within the context of the present invention, the organic solvent may be an alcohol, for example, methanol, ethanol, isopropanol, 1-butanol, 2-butanol, isoamyl alcohol, isobutyl alcohol, 1-pentanol, 1-propanol, 2-propanol, or mixtures thereof. One of skill in the art will recognize other alcoholic solvents that may be employed.

The solution may then be filtered, for example, through a Hyflo bed. The solution may then be cooled to a temperature of about 20° C. to about 35° C. Next, the solvent is removed for example, by distillation, spray drying, or agitated thin film drying (ATFD) to yield the amorphous form of afatinib dimaleate.

Another aspect of the present invention provides a premix solid dispersion of the amorphous form of afatinib dimaleate.

One embodiment of the present invention provides a process for the preparation of the premix solid dispersion of amorphous afatinib dimaleate which includes the following steps:
a) dissolving afatinib, maleic acid, and one or more pharmaceutically acceptable carriers in an organic solvent,
b) removing the organic solvent; and
c) isolating a solid dispersion of amorphous afatinib dimaleate.

According to this embodiment of the present disclosure, afatinib, maleic acid, and one or more pharmaceutical acceptable carriers are dissolved in an organic solvent. Within the context of the present invention, this step may be carried out at a temperature of about 50 to about 70° C. The organic solvent may be alcoholic, for example, methanol, ethanol, isopropanol, 1-butanol, 2-butanol, isoamyl alcohol, isobutyl alcohol, 1-pentanol, 1-propanol, 2-propanol and mixtures thereof. One of skill will recognize other alcoholic solvents that may be employed. The solution may then be filtered, for example, through a Hyflo bed. The solution may then be cooled to a temperature of about 20° C. to about 35° C. Next, the solvent is removed, which may be achieved for example, by distillation, spray drying, or agitated thin film dryer (ATFD), to yield a solid dispersion of amorphous afatinib dimaleate.

Within the context of the present disclosure, the one or more pharmaceutically acceptable carriers may be, for example, plasdone S-630 or polyvinylpyrrolidineK-30.

Another aspect of the present invention provides crystalline afatinib form-M. According to the present invention, crystalline afatinib dimaleate form-M may be characterized by a PXRD pattern having significant peaks at 2θ angle positions of about 6.64, 14.95, 17.71, and 18.98±0.2°.

Within the context of the present invention, crystalline afatinib dimaleate form-M may be further characterized by a PXRD pattern having significant peaks at 2θ angle positions of about 4.68, 6.64, 9.29, 10.55, 11.94, 13.38, 14.20, 14.95, 17.71, 18.11, 18.98, 20.38, 20.79, 21.25, 22.64, 23.31, 24.54, 25.67, 26.40, 27.86, 29.12, 30.35, 32.19, 33.31, 33.96, 36.65, 42.74, 44.84, and 46.30±0.2°.

One embodiment of the present invention provides a process for the preparation of crystalline afatinib form-M which includes the following steps:
a) dissolving afatinib in an organic solvent;
b) adding an anti-solvent; and
c) isolating crystalline afatinib form-M.

According to this embodiment of the present disclosure, afatinib is dissolved in an organic solvent. Within the context of the present invention, this step may be carried out at a temperature of about 40° C. to about 50° C. The organic solvent may be, for example, a ketone, an alcohol, or mixtures thereof. Examples of suitable ketones include acetone, methyl ethyl ketone, methyl isobutyl ketone, and ethyl isopropyl ketone. Examples of suitable alcohols include methanol, ethanol, isopropanol, 1-butanol, 2-butanol, isoamyl alcohol, isobutyl alcohol, 1-pentanol, 1-propanol, and 2-propanol. One of skill will recognize other ketone or alcoholic solvents that may be employed.

The solution may then be cooled to about 25° C. to about 30° C. Next, an anti-solvent is added and the solution may be stirred. Within the context of the present invention, the anti-solvent may be an ether solvent, a hydrocarbon solvent, water, or mixtures thereof. Suitable ether solvents include diethyl ether, di-isopropyl ether, and methyl tert-butyl ether. Suitable hydrocarbon solvents include n-hexane and n-heptane. One of skill in the art will recognize other ether solvents or hydrocarbon solvents that may be employed.

Crystalline afatinib form-M may then be isolated using methods well known in the art, including, for example, by filtering the solution and drying the solid at about 40° C. to about 50° C. under vacuum.

With all of the reactions disclosed above, one of skill in the art will recognize that the reaction conditions (e.g., reaction time or temperature) may be adjusted to achieve appropriate yield without undertaking undue experimentation and without departing from the scope of the present disclosure.

Another aspect of the present disclosure provides a pharmaceutical composition containing afatinib dimaleate and a pharmaceutically acceptable carrier. Within the context of the present invention, the afatinib dimaleate in the pharmaceutical composition may be crystalline or amorphous.

In some embodiments, the afatinib dimaleate polymorphs and amorphous afatinib dimaleate of the present invention may be included in tablets for oral administration. One of skill in the art will recognize a wide variety of pharmaceutically acceptable excipients that may be included in such a capsule formulation, including lactose monohydrate, microcrystalline cellulose, crospovidone, colloidal silicon dioxide, and magnesium stearate. The tablets may be coated with a film that may contain additional pharmaceutically acceptable excipients, for example, hypromellose, polyethylene glycol, titanium dioxide, talc, polysorbate 80, and artificial colorings.

The afatinib dimaleate polymorphs and amorphous forms and formulations disclosed herein may be included in formulations prescribed for the treatment of first-line treatment of patients with metastatic non-small cell lung cancer (NSCLC) whose tumors have epidermal growth factor receptor (EGFR) exon 19 deletions or exon 21 (L858R) substitution mutations as detected by an FDA-approved test. The afatinib dimaleate polymorphs and amorphous forms and formulations disclosed herein may be formulated as a solid dosage form, such as a tablet, containing about 20 mg to about 40 mg per capsule for administration to patients.

In view of the above description and the examples below, one of ordinary skill in the art will be able to practice the invention as claimed without undue experimentation. The foregoing will be better understood with reference to the following examples that detail certain procedures for the preparation of molecules, compositions and formulations according to the present invention. All references made to these examples are for the purposes of illustration. The following examples should not be considered exhaustive, but merely illustrative of only a few of the many aspects and embodiments contemplated by the present disclosure.

### EXAMPLE 1

### Preparation of Amorphous Afatinib Dimaleate

Afatinib dimaleate (10 g) was dissolved in methanol (300 mL) at 60-65° C. The clear solution was filtered through Hyflo to remove any undissolved particulate, cooled to 25-30° C. and subjected to spray-drying in a laboratory spray dryer (Model Buchi-290) with a feed rate of the solution 5-10 mL/min and an inlet temperature at 65° C. to yield amorphous form of afatinib dimaleate (6 g).

### EXAMPLE 2

### Preparation of Amorphous Afatinib Dimaleate

Afatinib (25 g) base and maleic acid (12.5 g) were dissolved in methanol (1125 mL) at 60-65° C. The clear solution was filtered through Hyflo to remove any undissolved particulate, cooled to 25-30° C. and subjected to spray drying in a laboratory spray dryer (Model Buchi-290) with a feed rate of the solution 5-10mL/min and an inlet temperature at 65° C. to yield amorphous form of afatinib dimaleate (30 g).

### EXAMPLE 3

### Preparation of Crystalline Afatinib Dimaleate Form-M1

Afatinib (12 g) was dissolved in N,N-dimethyl formamide (2 mL) at 65-70° C. To the clear solution maleic acid solution (0.5 g dissolved in 1 mL of N,N-dimethyl formamide at 65-70° C.) was added at 65-70° C. and cooled to 25-30° C. Methyl tert-butyl ether (20 mL) was added to the clear solution and stirred for 16 hours. The solid obtained was filtered and washed with methyl tert-butyl ether (2 mL) and dried at 40° C. under vacuum for 5-6 hours. The resulting product was identified as form-M1 of afatinib dimaleate (1.1 g).

### EXAMPLE 4

### Preparation of Crystalline Afatinib Dimaleate Form-M1

Afatinib (5 g) was dissolved in N,N-dimethyl formamide (10 mL) at 65-70° C. To the clear solution maleic acid solution (2.5 g dissolved in 5 mL of N,N-dimethyl formamide at 65-70° C.) was added at 65-70° C. and cooled to 25-30° C. Methyl tert-butyl ether (100 mL) was then added to the clear solution and stirred for 6 hours. The solid obtained was filtered and washed with methyl tert-butyl ether (10 mL) and dried at 40° C. under vacuum for 5-6 hours. The resulting product was identified as form-M1 of afatinib dimaleate (6 g).

### EXAMPLE 5

### Preparation of Amorphous Afatinib Dimaleate Premix

Afatinib (5 g), maleic acid (2.5 g) and Plasdone S-630 (3.75 g) was dissolved in methanol (250 mL) at 60° C. The clear solution was filtered through Hyflo to remove any undissolved particulate, cooled to 25-30° C. and subjected to spray drying in a laboratory spray dryer (Model Buchi-290) with feed rate of the solution 5 mL/min and inlet temperature at 70° C. to yield amorphous solid dispersion of afatinib dimaleate (6.5 g).

### EXAMPLE 6

### Preparation of Amorphous Afatinib Dimaleate Premix

Afatinib (5 g), maleic acid (2.5 g), and Plasdone S-630 (7.5 g) was dissolved in methanol (250 mL) at 60° C. The clear solution was filtered through Hyflo to remove any undissolved particulate, cooled to 25-30° C. and subjected to spray drying in a laboratory spray dryer (Model Buchi-290) with feed rate of the solution 5 mL/min and inlet temperature at 70° C. to yield amorphous solid dispersion of afatinib dimaleate (7.1 g).

### EXAMPLE 7

### Preparation of Amorphous Afatinib Dimaleate Premix

Afatinib (5 g), maleic acid (2.5 g) and Plasdone S-630 (15 g) was dissolved in methanol (250 mL) at 60° C. The clear solution was filtered through Hyflo to remove any undissolved particulate, cooled to 25-30° C. and subjected to spray drying in a laboratory spray dryer (Model Buchi-290) with feed rate of the solution 5 mL/min and inlet temperature at 70° C. to yield amorphous solid dispersion of afatinib dimaleate (8 g).

### EXAMPLE 8

### Preparation of Crystalline Afatinib Dimaleate Form-M1

Afatinib (6 g) and maleic acid (3 g) were dissolved in N,N-dimethyl formamide (18 mL) at 65-70° C., the clear solution was then cooled to 25-30° C. Slowly the material started precipitating and a thick mass was obtained after 5-10 minutes agitation at 25-30° C. To the resulting reaction mass, methyl-tert-butyl ether (120 mL) was added and stirred for 6 hours at 25-30° C. The free solid obtained was filtered and washed with methyl tert-butyl ether (12 mL) and dried at 40° C. under vacuum for 1-2 hours. The resulting product was identified as crystalline afatinib dimaleate form-M1.

### EXAMPLE 9

### Preparation of Crystalline Afatinib Dimaleate Form-M2

Crystalline afatinib dimaleate form-M1 (as prepared in Example 8) was dried under vacuum at 40° C. for 15-24 hours. The resulting product was identified as crystalline afatinib dimaleate form-M2.

### EXAMPLE 10

### Preparation of Crystalline Afatinib Dimaleate Form-M2

Crystalline afatinib dimaleate form-M1 (as prepared in Example 8) was dried under vacuum at 50° C. for 15 hours. The resulting product was identified as crystalline afatinib dimaleate form-M2.

### EXAMPLE 11

### Preparation of Crystalline Afatinib Dimaleate Form-M3

Afatinib (1 g) and maleic acid (0.5 g) were dissolved in 1-methyl 2-pyrrolidinone (3 mL) at 25-30° C. Acetone (20 mL) was added to the clear solution and stirred at 25-30° C. Slowly the material started to precipitate out after 15-20 minutes. The resulting suspension was further maintained under stirring at 25-30° C. for 18 hours. The obtained solid was filtered, washed with acetone (2 mL), and dried at 50° C. under vacuum for 3 hours. The resulting product was identified as crystalline afatinib dimaleate form-M3.

### EXAMPLE 12

### Preparation of Crystalline Afatinib Dimaleate Form-M4

Afatinib (5 g) was dissolved in methyl ethyl ketone (75 mL) at 25-30° C. Maleic acid solution (2.5 g of maleic acid dissolved in 25 mL of methyl ethyl ketone) was added to the clear solution at 25-30° C. and stirred. Initially a gummy residue was obtained which, upon ageing at 25-30° C. for 18 hours, resulted in a free solid. The solid obtained was filtered, washed with methyl ethyl ketone (10 mL), and dried at 40° C. under vacuum for 4 hours. The resulting product was Identified as crystalline afatinib dimaleate form-M4.

### EXAMPLE 13

### Preparation of Crystalline Afatinib Dimaleate Form-M5

Afatinib (1 g) was dissolved in acetone (10 mL) at 25-30° C. Maleic acid solution (0.5 g of maleic acid was dissolved in (5 mL) of acetone) was added to the clear solution at 25-30° C. and stirred for 18 hours. The obtained solid was filtered, washed with acetone (3 mL), and dried at 50° C. under vacuum for 4 hours. The resulting product was identified as crystalline afatinib dimaleate form-M5.

### EXAMPLE 14

### Preparation of Crystalline Afatinib Dimaleate Form-M6

Afatinib (1 g) was dissolved in acetone (20 mL) at 25-30° C. and cooled to 0-5° C. Maleic acid solution (0.5 g dissolved in 5 mL of acetone) was added to the clear solution at 0-5° C. and stirred for 4 hours. The obtained solid was filtered, washed with acetone (3 mL), and dried at 40° C. under vacuum for 3 hours. The resulting product was identified as form-M6 of afatinib dimaleate.

### EXAMPLE 15

### Preparation of Crystalline Afatinib Dimaleate Form-M6

Afatinib (3 g) was dissolved in acetone (90 mL) at 25-30° C. The clear solution was cooled to 0-5° C. and seeded with afatinib dimaleate form-M6 (30 mg). Maleic acid solution (1.5 g dissolved in 15 mL of acetone) was added to the reaction mass slowly at 0-5° C. and stirred for 6 hours. The obtained solid was filtered, washed with acetone (15 mL), and dried at 45° C. under vacuum for 16 hours. The resulting product was identified as form-M6 of afatinib dimaleate.

### EXAMPLE 16

### Preparation of Crystalline Afatinib Dimaleate Form-M6

Afatinib (2 g) was dissolved in acetone (60 mL) at 25-30° C. Seeds of afatinib dimaleate form-M6 (20 mg) was added to the clear solution. Maleic acid solution (1 g dissolved in 10 mL of acetone) was added slowly to the reaction mass at 25-30° C. and stirred for 18 hours. The obtained solid was filtered, washed with acetone (6 mL), and dried at 40° C. under vacuum for 3 hours. The resulting product was identified as form-M6 of afatinib dimaleate.

### EXAMPLE 17

### Preparation of Crystalline Afatinib Dimaleate Form-M6

Afatinib (2 g) was dissolved in acetone (80 mL) at 25-30° C. Seeds of afatinib dimaleate form-M6 (20 mg) was added to the clear solution. Maleic acid solution (1 g dissolved in 10 mL of acetone) was added to the reaction mass at 25-30° C. and stirred for 18 hours. The obtained solid was filtered, washed with acetone (6 mL), and dried at 40° C. under vacuum for 3 hours. The resulting product was identified as form-M6 of afatinib dimaleate.

### EXAMPLE 18

### Preparation of Crystalline Afatinib Dimaleate Form-M6

Afatinib (10 g) was dissolved in acetone (300 mL) at 25-30° C. The clear solution was cooled to 0-5° C. and seeded with afatinib dimaleate form-M6 (50 mg). Maleic acid solution (5 g dissolved in 50 mL of acetone) was added to the reaction mass slowly at 0-5° C. and stirred for 6 hours. The obtained solid was filtered, washed with acetone (50 mL), and dried at 45° C. under vacuum for 16 hours. The product obtained is milled and the resulting product was identified as form-M6 of afatinib dimaleate.

### EXAMPLE 19

### Preparation of Crystalline Afatinib Dimaleate Form-M7

Afatinib dimaleate crystalline form-M6 was heated to about at 80-90° C. for 1-2 hours using Bruker D8 variable temperature stage. The obtained product was identified as form-M7 of afatinib dimaleate.

### EXAMPLE 20

### Preparation of Crystalline Afatinib Dimaleate Form-M7

Afatinib dimaleate crystalline form-M6 was ground in a laboratory ball mill (Model: Retsch M-400) at a frequency (10 Hz) for 10-20 minutes. The obtained solid was identified as form-M7 of afatinib dimaleate.

### EXAMPLE 21

### Preparation of Crystalline Afatinib Dimaleate Form-M8

Afatinib (10 g) was dissolved in acetone (250 mL) at 25-30° C., filtered the solution through Hyflo and washed with acetone (50 mL). Maleic acid solution (5 g dissolved in 50 mL of acetone) was added to the clear solution slowly at 25-30° C. After half of the addition of maleic acid solution seeds of afatinib dimaleate form-M8 (100 mg) were added and the rest of the maleic acid solution was added. The obtained reaction mass was stirred for 24 hours at 25-30° C. The solid obtained was filtered, washed with acetone (50 mL), and dried at 50° C. under vacuum for 18 hours. The resulting product was identified as crystalline afatinib dimaleate form-M8.

### EXAMPLE 22

### Preparation of Crystalline Afatinib Dimaleate Form-M8

Afatinib (5 g) was dissolved in acetone (150 mL) at 25-30° C. The clear solution was cooled to 0-5° C. and seeded with afatinib dimaleate form-M8 (50 mg). Maleic acid solution (2.5 g dissolved in 25 mL of acetone) was added to the reaction mass slowly at 0-5° C. and stirred for 6 hours. The obtained solid was filtered, washed with acetone (15 mL), and dried at 40° C. under vacuum for 17 hours. The resulting product was identified as crystalline afatinib dimaleate form-M8.

### EXAMPLE 23

### Preparation of Crystalline Afatinib Dimaleate Form-M8

Afatinib dimaleate crystalline form-M6 was heated to about 40-50° C. for 30-60 minutes under nitrogen atmosphere using Bruker D8 variable temperature stage. The obtained product was identified as crystalline afatinib dimaleate form-M8.

### EXAMPLE 24

### Preparation of Crystalline Afatinib Dimaleate Form-M9

Afatinib (2 g) was dissolved in tetrahydrofuran (10 mL) at 25-30° C. Maleic acid solution (1.0 g dissolved in 5 mL of tetrahydrofuran) was added to the clear solution at 25-30° C. and stirred for 30 minutes. A gummy residue was obtained. Tetrahydrofuran (35 mL) was added to this residue and stirred for 3 hours at 25-30° C. The obtained solid was filtered, washed with tetrahydrofuran (10 mL), and dried at 40° C. under vacuum for 5 hours. The resulting product was identified as crystalline afatinib dimaleate form-M9.

### EXAMPLE 25

### Preparation of Crystalline Afatinib Dimaleate Form-M9

Afatinib (6 g) was dissolved in tetrahydrofuran (120 mL) at 25-30° C. and the solution was filtered to remove un-dissolved particles. Maleic acid solution (3.0 g dissolved in 60 mL of tetrahydrofuran) was added to the clear solution at 25-30° C., stirred for 1 hour, then cooled to 0-5° C., and stirred for 1 hour more. The obtained solid was filtered, washed with tetrahydrofuran (30 mL), and dried at 50° C. under vacuum for 18 hours. The resulting product was identified as crystalline afatinib dimaleate form-M9.

### EXAMPLE 26

### Preparation of Crystalline Afatinib Dimaleate Form-M9

Afatinib (6 g) was dissolved in tetrahydrofuran (120 mL) at 25-30° C. and the solution was filtered to remove un-dissolved particles. The clear solution was seeded with afatinib dimaleate form-M9 (50 mg) then added maleic acid solution (3.0g dissolved in 60 mL of tetrahydrofuran) at 25-30° C. for 60 minutes, and stirred for 20 hours. The solid obtained was filtered, washed with tetrahydrofuran (30 mL) and dried at 50° C. under vacuum for 18 hours. The resulting product was identified as crystalline afatinib dimaleate form-M9.

### EXAMPLE 27

### Preparation of Crystalline Afatinib Dimaleate Form-M9

Afatinib (3 g) was dissolved in tetrahydrofuran (60 mL) at 25-30° C. and filtered the solution to remove un-dissolved particles. Taken clear filtrate into round bottom flask under nitrogen atmosphere and seeded with afatinib dimaleate form-M9 (100 mg), then added maleic acid solution (1.5 g dissolved in 30 mL of tetrahydrofuran) at 25-30° C. for 40 minutes, and stirred for 20 hours under nitrogen atmosphere. The solid obtained was filtered, washed with tetrahydrofuran (15 mL), and dried at 50° C. under vacuum for 18 hours. The resulting product was Identified as crystalline afatinib dimaleate form-M9.

### EXAMPLE 28

### Preparation of Crystalline Afatinib Form-M

Afatinib (1 g) was dissolved in methyl isobutyl ketone (6 mL) at 50° C., filtered the solution to remove un-dissolved particles. Taken clear filtrate, cool to 25-30° C. added methyl tert-butyl ether (30 mL) at same temperature and stirred for 16 hours. The solid obtained was filtered, washed with methyl tert-butyl ether (2 mL) and dried at 45° C. under vacuum for 6 hours. The resulting product was identified as form-M of afatinib.

### EXAMPLE 29

### Preparation of Crystalline Afatinib Form-M

Afatinib (1 g) was dissolved in methyl isobutyl ketone (6 mL) at 50° C., filtered the solution to remove un-dissolved particles. Taken clear filtrate, cool to 25-30° C. added n-heptane (30 mL) at same temperature and stirred for 15 hours. The solid obtained was filtered, washed with butyl n-heptane (3 mL), and dried at 45° C. under vacuum for 6 hours. The resulting product was identified as form-M of afatinib.

### EXAMPLE 30

### Preparation of Crystalline Afatinib Form-M

Afatinib (1 g) was dissolved in acetone (5 mL) at 40° C., filtered the solution to remove un-dissolved particles. Taken filtrate heated to 40° C. to get clear solution, then cool to 25-30° C. added water (15 mL) at same temperature and stirred for 16 hours. The solid obtained was filtered, washed with water (2 mL) and dried at 45° C. under vacuum for 6 hours. The resulting product was Identified as form-M of afatinib

### EXAMPLE 31

### Preparation of Crystalline Afatinib Form-M

Afatinib (1 g) was dissolved in acetone (5 mL) at 40° C., filtered the solution to remove un-dissolved particles. Taken filtrate heated to 40° C. to get clear solution, then cool to 25-30° C. added methyl tert-butyl ether (30 mL) at same temperature and stirred for 16 hours. The solid obtained was filtered, washed with methyl tert-butyl ether (5 mL) and dried at 45° C. under vacuum for 6 hours. The resulting product was identified as form-M of afatinib.

### EXAMPLE 32

### Preparation of Crystalline Afatinib Form-M

Afatinib (1 g) was dissolved in acetone (5 mL) at 40° C., filtered the solution to remove un-dissolved particles. Taken filtrate heated to 40° C. to get clear solution, then cool to 25-30° C. added n-heptane (30 mL) at same temperature and stirred for 16 hours. The solid obtained was filtered, washed with n-heptane (5 mL) and dried at 45° C. under vacuum for 6 hours. The resulting product was identified as form-M of afatinib.

### EXAMPLE 33

### Preparation of Crystalline Afatinib Form-M

Afatinib (70 g) was dissolved in methanol (250 mL) at 25-30° C., filtered the solution through high-flow bed to remove un-dissolved particles, and washed bed with methanol (30 mL). The clear filtrate was placed into round bottomed flask, cooled to 25-30° C., added water (1120 mL), and stirred at same temperature for 6 hours. The solid obtained was filtered, washed with water (100 mL) and dried at 50° C. under vacuum for 16 hours. The resulting product was identified as form-M of afatinib.

We claim:

1) Crystalline afatinib dimaleate form-M8.

2) The crystalline afatinib dimaleate form-M8 according to claim 1, having a powder X-ray diffraction pattern having 2θ angle positions of significant peaks at about 5.54, 17.17, 17.50, 18.91, 19.34, 21.43, and 22.60±0.2°.

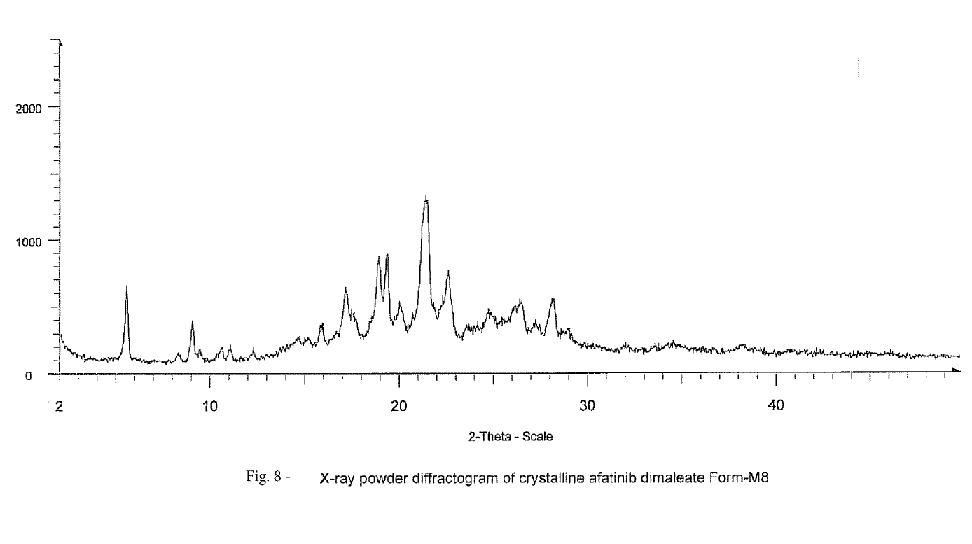

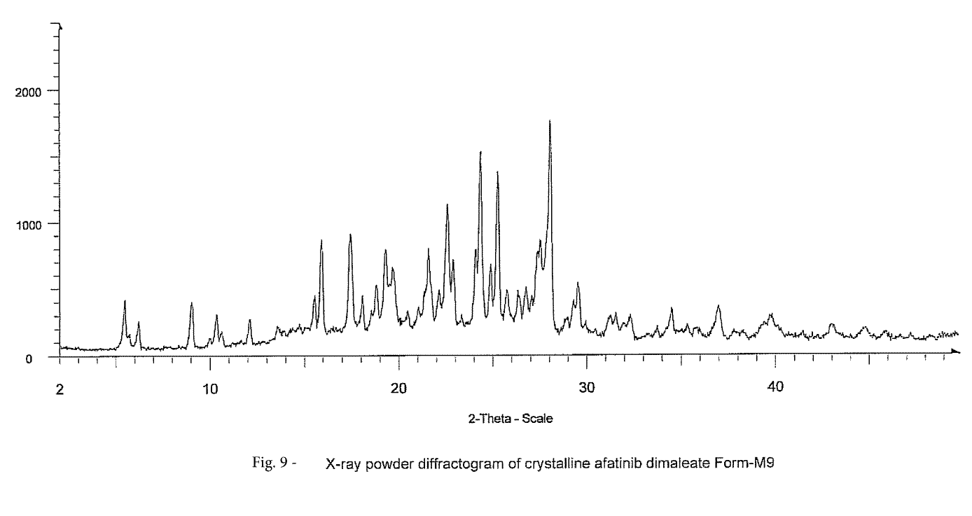

3) The crystalline afatinib dimaleate form-M8 according to claim 1, having an X-ray diffraction pattern as shown in FIG. 8.

4) A process for preparation of crystalline afatinib dimaleate form-M8, comprising the steps of:
a) dissolving afatinib in an organic solvent;
b) optionally seeding with afatinib dimaleate form-M8;
c) adding maleic acid solution; and
d) isolating crystalline afatinib dimaleate form-M8 by filtration, washing, and drying.

5) The process according to claim 4, wherein the organic solvent is selected from acetone.

6) A process for the preparation of crystalline afatinib dimaleate form-M8, comprising heating crystalline afatinib dimaleate form-M6 at about 40-50° C. for about 30-60 minutes.
